(19) United States
(12) Patent Application Publication
(10) Pub. No.: US 2009/0099464 A1
(43) Pub. Date: Apr. 16, 2009

(54) METHOD FOR DETERMINING CARDIOVASCULAR PARAMETERS AND DEVICE AND COMPUTER PROGRAM PRODUCT FOR CARRYING OUT SAID METHOD

(75) Inventors: Stefan Hollinger, Kronberg (DE); Dieter Wunder, Schotten (DE); Ulrich Heck, Krefeld (DE); Dirk Freund, Kelkheim (DE); Brigitte Harttmann, Niedernhausen (DE); Gerrit Roenneberg, Darmstadt (DE); Fred Schnak, Kronberg (DE)

Correspondence Address: DARBY & DARBY P.C., P.O. BOX 770, Church Street Station, New York, NY, 10008-0770, US

(73) Assignee: Kaz USA, Inc., Southborough, MA (US)

(21) Appl. No.: 12/249,346

(22) Filed: Oct. 10, 2008

Related U.S. Application Data
(63) Continuation of application No. 11/910,409, now abandoned.

(30) Foreign Application Priority Data
Apr. 1, 2005 (DE) 102005014950.2

(51) Int. Cl. A61B 5/02

(52) U.S. Cl. 600/490; 600/481

(57) ABSTRACT

A method, device and computer program product used to determine at least one cardiovascular parameter P^{m}, for example, the pulse, average arterial pressure (MAP), systolic pressure, diastolic pressure, arterial wave form, speed of the blood flow, systole duration, or diastole duration, by a measuring device which detects, in a non-invasive manner and in a time interval, several signals Fₗ^{m} of an artery, through which blood flows, as a reaction to an application of force to the artery. As a result, distances between the signals Fₗ^{m} and the reference signals are defined from the detected signals Fₗ^{m} with the aid of reference signals, which are used to determine the at least one parameter P^{m}

### CROSS-REFERENCE TO PRIOR APPLICATION

This application is a continuation of U.S. National Phase application Ser. No. 11/910,409, filed on Oct. 1, 2007 under 35 U.S.C. §371 of International Application No. PCT/EP2006/002207, filed on Mar. 10, 2006, and claims the benefit of German Patent Application No. 102005014950.2, filed on Apr. 1, 2005, both of which are incorporated herein. The International Application was published in German on Oct. 5, 2006 as WO 2006/102976 A1 under PCT Article 221(2).

### FIELD OF THE INVENTION

The invention relates to a method for determining at least one cardiovascular parameter. Furthermore, the invention relates to a device and to a computer program product for carrying out the method.

### BACKGROUND

A method for determining cardiovascular parameters has been developed, for example, by the Cardiodynamics Company of San Diego, USA. With this method, a sensor transmits an electrical signal through the thorax of the human body. The electrical impedance of the thorax is measured as a reaction to this signal. Since the volume and the velocity of the blood in the aorta vary with each heart beat, there are also fluctuations in the impedance of the thorax. The fluctuations of these impedance values can be used for determining different cardiovascular parameters.

However, these parameters are obtained by noninvasive impedance measurements, which can be carried out on the thorax of the patient only at great expense for equipment. An ambulant determination of the cardiovascular parameters is therefore not possible.

### SUMMARY OF THE INVENTION

The present invention is therefore based on the objective of indicating a method, a device and a computer program product for determining at least one cardiovascular parameter, with which a comprehensive cardiovascular diagnosis can be carried out easily without a major expense for equipment.

The present invention is for a method for determining at least one cardiovascular parameter, and a device and computer program for carrying out the method. The invention includes applying a force to an artery, through which blood is flowing. Signals produced as a reaction to the force on the artery are noninvasively detected in a time interval using a measuring instrument. Using intervals between the detected signals and reference signals, the cardiovascular parameter is determined.

### DETAILED DESCRIPTION

For a method of this type, intervals between the signals Fₗ^{m} and the reference signal, which can be used to ascertain the at least one parameter P^{m}, are determined from the detected signals Fₗ^{m} with the help of reference signals. In this connection, 1 extends from 1 to Lₘ, Lₘ being the number of signals, which is equal to or greater than the number M of the parameters P^{m}, which are to be determined.

As a result of the invention, it is possible to determine cardiovascular parameters on the basis of time-resolved pulsation signals, the cause of which is the pulsatile flow in the arteries. In this connection, it is, to begin with, of subordinate importance whether or not the signal used corresponds to the inner arterial pressure. A strong correlation of the signal with the pressure is of far greater importance. By means of the invention, it is possible to draw conclusions concerning the cardiovascular parameters from the properties of the signals recorded. On the basis of functional relationships, it is also possible to use signals, which are linked to the flow velocity of the blood in the veins or to the rate of spreading of the pressure waves and velocity waves in the veins it is important to produce a defined relationship between properties of the signal recorded and cardiovascular parameters, reference values being assigned to the signals detected. In an implementation of the method, this functional relationship is entered into a device, for example, a product of a computer system. In this connection, it has proven to be advantageous to define the functional relationship in such a manner that the parameters (which are to be determined, follow as a value from the (measurable) signals as input. In order to avoid that the cardiovascular parameter determination is underdetermined, at least as many signals as parameters are used.

According to one embodiment of the present invention, a reference database F_{l,m,k}^{Ref} is provided as a reference, which is used to define the functional relationship and is stored in the reference measurements, for which a linkage between signals and parameters is known. Moreover, k ranges from 1 to K, K being the number of reference measurements. Clinical measurements, for which, with a suitable device, pulsatile signals in man are recorded, which are related to the arterial pulsation, may form the basis for this database. At the same time, the parameters, which are to be determined, are determined with one or more reference methods. In this way, a relationship is brought about between signals and parameters. A comparison or an assignment of the measured values with parameters of the database is thus possible.

Moreover, an interval function \tilde{D}_{k,m}(Fₗ^{m},F_{l,m,k}^{Ref}) may be defined using the reference database F_{l,m,k}^{Ref}. Evidently, the database covers the signal space as well as the parameter space in discrete form. Accordingly, the functional relationship between signals/parameters is defined only on a multidimensional lattice, the dimensions of which correspond to the number of signals. The points in this lattice generally are not distributed equidistantly in the signals space. If a measurement is made, the signal point measured generally does not coincide with a lattice point. By means of the functional relationship however, a value of the reference database can be assigned to each signal, for example, by interpolation.

For example, the functional relationship may be represented as:

$${P}^{m} = \frac{{\sum }_{k = 1}^{K} {\tilde{D}}_{k , m} \cdot {P}_{k}^{m}}{{\sum }_{k = 1}^{K} {\tilde{D}}_{k , m}} .$$

Pₖ^{m} being read from the reference data base. By these means, parameter values can also be assigned on the basis of signal points between the lattice points. This is accomplished by the interval function \tilde{D}_{k,m}.

Optionally, only (time) intervals, which are smaller than a specified interval or a time interval, are used for determining the parameters P^{m} This can be accomplished by limiting conditions \tilde{D}_{k,m,grenz}, which can be determined empirically and for which \tilde{D}_{k,m}≦\tilde{D}_{k,m,grenz}. By these means, the computational effort and the time required for determining the parameters is minimized appreciably, since, a priori, only data, which is similar to the measurement investigated, is selected from the reference database.

Conventional measuring instruments may be used to determine cardiovascular parameters. With these instruments, for example, the reaction of an artery, through which blood is flowing, to an applied force, such as a pressure, is detected. In the next step, due to the invention, fewer measurements have to be analyzed than were originally recorded. By these means, further processing of measurements, which are not meaningful for physiological reasons, is excluded.

According to a further concept of the invention, the intervals of the signals detected and of the reference signals are reported. For example, the interval function

$${\tilde{D}}_{k , m} = \sqrt{{\sum }_{l = 1}^{{L}_{m}} {( \frac{{F}_{l}^{m} - {F}_{l , m , k}^{Ref}}{{max}_{k} ( {F}_{l , m , k}^{Ref} ) - {min}_{k} ( {F}_{l , m , k}^{Ref} )} )}^{2}}$$

can be used. Mathematically, this interval function can be solved easily and is therefore suitable particularly for pre-selecting signals necessary for determining the cardiovascular parameters.

According to another embodiment, the time-resolved signals Fₗ^{m}, as well as the reference database F_{l,m,k}^{Ref}, present in a particular time resolution, are scaled in terms of time. This can be represented, for example, as Fₗ^{m} [symbol] M(tₗ) and F_{l,m,k}^{Ref} [symbol] Mₖ(tₗ). The inverse of the heart rate is suitable particularly for scaling in terms of time. With that, the cardiovascular parameters, which are to be determined, are obtained in a form independent of time.

From these time-scaled signals and the time-scaled reference database, a new interval function D_{k,m} is defined and used for the determination of time-scaled parameter P^{m}. For example,

$${P}^{m} = \frac{{\sum }_{k = 1}^{K} {w}_{m} ( {D}_{k , m} ) \cdot {P}_{k}^{m}}{{\sum }_{k = 1}^{K} {w}_{m} ( {D}_{k , m} )}$$

$$in which$$

$${w}_{k} ( {D}_{k , m} ) = \{ \begin{matrix}{w}_{k} ( {D}_{k , m} ) & if & {D}_{k , m \leq } {D}_{k , m , limit} \\ 0 & otherwise & \end{matrix}$$

representing a weighting function where, for example

$${D}_{k , m} = \sqrt{{\sum }_{i = {s}_{m}}^{{e}_{m}} {( {M}_{k} ( {t}_{i} ) - M ( {t}_{i} ) )}^{2}}$$

where eₘ≧1, N≧sₘ and N represents the number of times samples. The indexes sₘ and eₘ used are used here for selecting a particular time window (for example, the systolic increase in the pressure curve or the diastolic decrease in the pressure curve) in the signal for the determination of the parameter P^{m}. The signals of the database measured are present with a certain time resolution.

As already described, it is possible that the signals are stored in a time-scaled formed in the database. As already indicated, especially the inverse of the heart rate is suitable as a timescale. The new interval quantities D_{k,m} are calculated with the help of these scaled measurements Mₖ(tₗ) (k: index for the selected reference measurements in the database, l: index for the discretization of the time-resolved signals) and M(tₗ) (measurement to be investigated).

According to another embodiment of the invention, the following polynomials of any order Nₘ can be used for determining the parameters P^{m}:

$${P}^{m} = {\sum }_{{l}_{1} = 0}^{{L}_{m}} ⋯ {\sum }_{{l}_{{N}_{m}} = 0}^{{L}_{m}} {C}_{{l}_{1} , … , {l}_{{N}_{m}} , m} {F}_{{l}_{1}}^{m} \cdot … \cdot {F}_{{l}_{{N}_{m}}}^{m} with {F}_{0}^{m} = 1.$$

For this purpose, a certain number of measurements are assigned, once again by an interval function, to the reference measurements in the database. The polynomials of any order Nₘ, which produce a local relationship between the measured data and the parameters in the vicinity of the measurement, which is to be evaluated, are ascertained on the basis of this reference measurement. Linear functions:

$${P}^{m} = ( {C}_{0 , m} {C}_{1 , m} {C}_{2 , m} ⋯ {C}_{{L}_{m} , m} ) \cdot ( \begin{matrix}1 \\ {F}_{1}^{m} \\ {F}_{2}^{m} \\ ⋮ \\ {F}_{{L}_{m}}^{m}\end{matrix} )$$

or quadratic polynomials

$${P}^{m} = ( 1 {F}_{1}^{m} {F}_{2}^{m} ⋯ {F}_{{L}_{m}}^{m} ) \cdot ( \begin{matrix}{C}_{0 , 0 , m} & {C}_{1 , 0 , m} & {C}_{2 , 0 , m} & ⋯ & {C}_{{L}_{m} , 0 , m} \\ & {C}_{1 , 1 , m} & {C}_{0 , 0 , m} & ⋯ & {C}_{{L}_{m} , 1 , m} \\ & & {C}_{2 , 2 , m} & ⋯ & {C}_{{L}_{m} , 2 , m} \\ & & & ⋱ & ⋮ \\ symmetrisch & & & & {C}_{{L}_{m} , {L}_{m} , m}\end{matrix} ) \cdot ( \begin{matrix}1 \\ {F}_{1}^{m} \\ {F}_{2}^{m} \\ ⋮ \\ {F}_{{L}_{m}}^{m}\end{matrix} )$$

can be used as the simplest polynomials. From the system of these equations, the coefficients of the polynomials can be determined by known methods. For this purpose, the number of signals used must be at least equal to the number of parameters, as otherwise the problem of the cardiovascular parameter determination is under-determined.

The device according to the invention has at least one data processing installation, especially a microprocessor, at least one memory unit and at least one measuring instrument, especially a blood pressure measuring instrument, for detecting signals from an artery, through which blood is flowing. Such a device has all the necessary instruments and implements, which are necessary for carrying out the inventive method.

In order to be able to carry out a simple, rapid and efficient determination of at least one cardiovascular parameter, the instrument may be formed for measuring at an upper arm, a wrist or a finger. Other superficial measurements, that is, ones which are not accessible inresively, are also possible. Consequently, medical lay persons are also in a position to carry out the determination of the cardiovascular parameters accurately, since the handling of the device is simplified.

In this respect, the memory unit and the measuring device can be disposed in a common housing. As a result of this measure, the device as a whole has an extremely compact construction, so that it requires only a little space. By these means, the determination of the cardiovascular parameters over a longer period of time is simplified, since the device, due to its compact construction, can also be transported easily and therefore can be taken along by the user even when traveling and the data measured can be stored in the memory unit.

According to another embodiment of the invention, the memory unit can be formed for storing several reference databases. The different reference databases can take different personal parameters into consideration, so that the device can also be used for different persons or adapted to their personal relationships.

According to another embodiment of the invention, the device can have an interface for exchanging data. Accordingly, the parameters measured can be stored in a further, larger memory over a longer period of time, so that comparison data can be collected over a longer period of time.

A computer program product can also be used to carry out the method. The computer program product can be stored on a computer system, on which it can be run.

Two examples of the invention are described in the following.

### FIRST EXEMPLARY EXAMPLE

In one embodiment of the invention, properties of the signal or signals, which are recorded, are to be determined. These properties may be properties of the signals in the time space and/or in the frequency space. In particular, values, slopes and/or curvatures at particular times and integrations of these quantities over certain time intervals, with or without a window function, must be mentioned (this corresponds to an averaging and filtering of the corresponding quantities). Time intervals between certain points of the signals can also be used, especially those between the starting points of two consecutive heart beats (RR interval), as well as the duration of systoles and diastoles.

Complete or incomplete results of a harmonic analysis—frequencies (especially heart rates), amplitudes and phases—can be used as signals in the frequency space. Methods other than the harmonic analysis can also be used for the frequency analysis, for example, those which use elapsed time as a basis instead of the trigonometric functions and which come closer to course of the pulsatile pressure or velocity in the artery than do the trigonometric functions.

In a further step, the signals are used for selecting from the database the reference measurement measurements, which are similar to the measurement that is to be investigated. For this purpose, selected quantities are introduced, such as a distance measure in the signal space

$${\tilde{D}}_{k , m} = \sqrt{{\sum }_{l = 1}^{{L}_{m}} {( \frac{{F}_{l , m}^{Mess} - {F}_{l , m , k}^{Ref}}{{max}_{i} ( {F}_{l , m , k}^{Ref} ) - {min}_{k} ( {F}_{l , m , k}^{Ref} )} )}^{2}} , \qquad Equation 1$$

which gives a measure of the distance between the measurement, which is to be investigated, and the k^{th} reference measurement for the determination of the m^{th} parameter. For the further determination of the parameter P^{m}, only those reference measurements are used, the selected quantities of which, that is, for example, the distances \tilde{D}_{k,m} fulfill a certain condition, such as \tilde{D}_{k,m}23 \tilde{D}_{grenz}, that is, for example, are not more than a certain distance from the measurement, which is to be investigated.

The advantages of a pre-selection are seen to lie in that, in the next computationally intensive step, fewer measurements from the database have to be analyzed. Likewise, further processing of measurements of the database, which are not comparable, for physiological reasons, with the measurement to be investigated, is excluded. In addition, ambiguities in the relationship between signals and parameters can be handled, provided that this ambiguity no longer exists in the partial quantities selected.

The actual time-resolved signals of the measurement to be investigated and of the database exist with a certain time resolution. It is likewise possible to store the signals in a time-scaled form in the database. In particular, the inverse of the heart rate can be stored as a timescale.

In a first step for determining the parameters, the signals are scaled on the ordinates as well as on the abscissas of the signals, in order to make them comparable. Interval quantities are calculated once again with the help of these scaled measurements Mₖ(tₖ) (k: index for the above selected reference measurements in the database, l: index for the discretization of the time-resolved signals) and M(tₗ) (measurement, to be investigated), an interval measure being used. One possibility for using this measure is:

$${D}_{k , m} = \sqrt{{\sum }_{i = {s}_{m}}^{{e}_{m}} {( {M}_{k} ( {t}_{i} ) - M ( {t}_{i} ) )}^{2}} , \qquad Equation 2$$

where sₘ, eₘ (with N≧sₘ, eₘ≧1) are indexes for selecting a certain time window (such as the systolic increase in the pressure curve, the diastolic decrease in the pressure curve) in the signal for the determination of the parameter Pₘ and N represents the number of time samples of the complete signal, which have been used. In the last step, the parameters sought are determined. For this purpose, the corresponding parameters Pₘ^{k} (k: index for the reference measurements in the database, selected above, m: index for the parameter sought) are read from the database for each parameter Pₘ sought. Pₘ is calculated with the interval measures D_{k,m} by weighting.

$${P}^{m} = \frac{{\sum }_{k = 1}^{K} {w}_{m} ( {D}_{k , m} ) \cdot {P}_{k}^{m}}{{\sum }_{k = 1}^{K} {w}_{m} ( {D}_{k , m} )} . \qquad Equation 3$$

The weighting functions wₘ(D_{k,m}), introduced in Equation 3, depend on the interval measure D_{k,m} and can be different for the parameters P^{m}, which are to be determined. The dependence on the interval measure may, for example, be inversely proportional to the square of the interval. However, situations are also conceivable, in which the inclusion of reference measurements of the database, which are far removed from the measurement to be investigated, may not be entered into the calculation. This can be brought about with the help of the weighting functions in that

$${w}_{k} ( {D}_{k , m} ) = \{ \begin{matrix}{w}_{k} ( {D}_{k , m} ) & if & {D}_{k , m} \leq {D}_{k , m , grew} \\ 0 & otherwise & \end{matrix} \qquad Equation 4$$

is selected with a random weighting function in the range D_{k,m}≦D_{k,m,grenz}. Likewise, it is also possible that the weighting functions depend on the selected quantities \tilde{D}_{k,m}. With that, the parameters sought are determined unambiguously.

### SECOND EXEMPLARY EMBODIMENT

For this embodiment, the parameters are defined as in the first embodiment.

In a second step, an interval measure of the above definition is also required for the parameter determination. It is used for determining a certain number M of measurements in the database of the reference measurements, for example, which of the measurements, for which the parameters are to be determined, lie closest in the sense of the interval measure used.

On the basis of these M reference measurements, multi-dimensional fit functions are determined (for each parameter) and produce a higher local relationship between the measured data and the parameters in the vicinity of the measurement, which is to be evaluated. These fit functions may, for example, be linear

$$\begin{matrix}{P}^{m} = ( {C}_{0 , m} {C}_{1 , m} {C}_{2 , m} ⋯ {C}_{{L}_{m} , m} ) \cdot ( \begin{matrix}1 \\ {F}_{1}^{m} \\ {F}_{2}^{m} \\ ⋮ \\ {F}_{{L}_{m}}^{m}\end{matrix} ) \\ quadratic & Equation 5 \\ {P}^{m} = ( 1 {F}_{1}^{m} {F}_{2}^{m} ⋯ {F}_{{L}_{m}}^{m} ) \cdot ( \begin{matrix}{C}_{0 , 0 , m} & {C}_{1 , 0 , m} & {C}_{2 , 0 , m} & ⋯ & {C}_{{L}_{m} , 0 , m} \\ & {C}_{1 , 1 , m} & {C}_{0 , 0 , m} & ⋯ & {C}_{{L}_{m} , 1 , m} \\ & & {C}_{2 , 2 , m} & ⋯ & {C}_{{L}_{m} , 2 , m} \\ & & & ⋱ & ⋮ \\ symmetrisch & & & & {C}_{{L}_{m} , {L}_{m} , m}\end{matrix} ) \cdot ( \begin{matrix}1 \\ {F}_{1}^{m} \\ {F}_{2}^{m} \\ ⋮ \\ {F}_{{L}_{m}}^{m}\end{matrix} ) & Equation 6\end{matrix}$$

or polynomial of any order Nₘ

$${P}^{m} = {\sum }_{{l}_{1} = 0}^{{L}_{m}} ⋯ {\sum }_{{l}_{{N}_{m}} = 0}^{{L}_{m}} {C}_{{l}_{1} , … , {l}_{{N}_{m}} , m} {F}_{{l}_{1}}^{m} \cdot … \cdot {F}_{{l}_{{N}_{m}}}^{m} with {F}_{0}^{m} = 1 \qquad Equation 7$$

in which there are signals Fₖ^{m}. The determination of the numbers C_{l,m}, C_{i,d,m},C_{h...l,m}, that is, of the coefficients of the polynomials, is carried out with relevant, known methods.

We claim:

1. A method for determining at least one cardiovascular parameter P^{m}; comprising;
applying a force to artery, through which blood is flowing;
noninvasively detecting signals Fₗ^{m} in a time interval as a reaction to the force on the artery using a measuring instrument; and
using intervals between the signals Fₗ^{m} and reference signals to determine the at least one parameter P^{m}, the intervals being defined from the detected signals Fₗ^{m} in combination with the reference signals.

2. The method of claim 1, wherein a reference database F_{l,m,k}^{Ref} is used as reference.

3. The method of claim 2, wherein and interval function \tilde{D}_{k,m}(Fₗ^{m},F_{l,m,k}^{Ref}) is defined using the reference database F_{l,m,k}^{Ref}.

4. The method of claim 1 wherein the distances of reference measurements from the measurement to be analyzed, are less than a specified distance.

5. The method of claim 1 wherein at least one of the distances of the signals detected and the reference signals is averaged.

6. The method of claim 1 wherein the signals Fₗ^{m} and the reference database F_{l,m,k}^{Ref}, are time-scaled in a particular time resolution.

7. The method of claim 6 wherein a new interval function D_{k,m}, is defined from the time-scaled signals and the time-scaled reference database and used for determining time-scaled parameters P^{m}.

8. The method of claim 1 wherein polynomials of any order Nₘ are used for determining the parameters Pₘ.

9. A device for determining at least one cardiovascular parameter Pₘ comprising:
at least one data processing installation, at least one memory unit;
at least one measuring instrument for detecting signals of an artery, through which blood is flowing; and
at least one data processing installation configured to determine the parameter based on intervals between the signals and the reference signals.

10. The device of claim 9 wherein the measuring instrument is configured to measure at least one of an upper arm, a wrist and a finger.

11. The device of claim 9 wherein the data processing installation, the memory unit and the measuring instrument are disposed in a common housing.

12. The device of claim 9 further comprising an interface for exchanging data is provided.

13. The device of claim 9 wherein the memory unit is configured to storing several reference databases.

14. A computer program product for determining at least one cardiovascular parameter P^{m}, such as a pulse, by means of the arterial pressure (MAP), the systolic pressure, the diastolic pressure, the vein elasticity, the peripheral resistance and the like, for carrying out a method of claim 1.
